(19) United States
(12) Patent Application Publication
(10) Pub. No.: US 2015/0018933 A1
(43) Pub. Date: Jan. 15, 2015

(54) AORTIC STENT-GRAFT

(71) Applicant: BEIJING PERCUTEK THERAPEUTICS CO., LTD., Beijing (CN)

(72) Inventors: Fan Yang, Beijing (CN); Wei Zheng, Beijing (CN); Jiahua Xiao, Beijing (CN)

(21) Appl. No.: 14/369,650

(22) PCT Filed: Nov. 23, 2012
(86) PCT No.: PCT/CN2012/085211

§ 371 Date: Jul. 21, 2014

(30) Foreign Application Priority Data
Dec. 29, 2011 (CN) 201120564767.9
Dec. 29, 2011 (CN) 201120565138.8

(51) Int. Cl. A61F 2/07; A61F 2/06

(52) U.S. Cl. CPC A61F 2/07; A61F 2/064; A61F 2220/0016
USPC 623/1.14

(57) ABSTRACT

An aortic stent-graft capable of avoiding migration of location, which includes a covered stent body and a bare stent connected to a proximal end of the covered stent body, with barbs for looseness-proof fixation which are provided at each wave crest and each wave trough of the bare stent and extend towards a distal end of the covered stent body. After the aortic stent-graft is fastened by the looseness-proof fixation structures, because the barbs arranged at each wave crest of the bare stent are not on the same plane with the barbs arranged at each wave trough of the bare stent, simultaneous relative motions of the two sets of barbs which may lead to detachment or loosening of the looseness-proof fixation structures will not happen, so that the aortic stent-graft is able to be fastened onto a blood vessel wall more firmly.

### TECHNICAL FIELD

The present invention relates to medical apparatus and instruments, and especially relates to an aortic stent-graft.

### BACKGROUND OF THE INVENTION

In the present invention, the term “distal end” refers to the end that is away from the heart along the blood flow direction, and the term “proximal end” refers to the end that is near the heart along the blood flow direction.

Aortic aneurysm refers to local or disseminated abnormal dilation of aortic wall, which compresses surrounding organs and causes symptoms, with a major danger of aneurismal rupture. Aortic aneurysm often occurs in the ascending aorta, the aortic arch, the thoracic descending aorta, the thoraco-abdominal aorta and the abdominal aorta. Traditional surgical treatment methods are thoracotomy and laparotomy operations to excise aortic lesions and to replace the lesions with artificial blood vessels, these operations have great surgical risks, while a lot of aortic lesions are not able to be surgically treated, and the mortality rate of these surgical operations are high, with a tendency to easily cause severe complications of functional insufficiency of heart, brain or kidney. In the 1990s, since the emerging of the method of endovascular graft exclusion of abdominal aortic aneurysm of the human body, it has quickly become the first choice of therapeutic method for treating aortic aneurysm.

To perform an endovascular graft exclusion of aortic aneurysm, firstly, imageological diagnosis such as CT arteriography is performed on a patient with aortic aneurysm to acquire precise data of the aortic aneurysm, then, an aortic stent-graft with suitable diameter and length is selected or made according to the acquired data. During the operation, a small incision of 3 cm long is made at the groin, and under surveillance of X-ray monitoring, a stent-graft is delivered to the location of aortic lesion through the femoral artery by a corresponding delivery system, then the stent-graft is released from a catheter. The released aortic stent-graft expands to its original diameter and is conformed to the aortic wall by its own elastic force to cover the portion of aortic lesion, thus the stent-graft creates a new blood passageway within the diseased blood vessel, blocks the aortic lesion from fresh blood supply and makes the aortic aneurysm to gradually form thrombus, thereby, the compressing stress of the aortic aneurysm on surrounding tissues and organs is relieved, and the danger of massive hemorrhage caused by rupture of the aortic aneurysm is eliminated.

As the endovascular graft exclusion is operated through a blood vessel far from the location of aortic lesion, it avoids collateral damage resulted from anatomy of the diseased portion; and during the operation, the blocking time of blood flow is extremely short, which avoids complications of viscera organ ischemia; moreover, endovascular treatment of most central type blood vessels is able to be carried out under the condition of small incision with topical anesthesia, which lowers the requirements of anesthesia, greatly reduced operation wound, and speeds up post-operation recovery. These advantages make it the first choice of therapeutic method for treating aortic aneurysm, however, presently there are still certain problems with this kind of endovascular graft exclusion of aortic aneurysm, for example, position-shifting of the stent-graft is likely to occur, which is a reason of causing failure of treatment and future complications, and the problem may be solved by selection of appropriate stent-graft or improvement of the stent-graft.

The patent literature W02004002370A1 discloses a thoracic aortic stent-graft with barbs, comprising a tubular graft material, zigzag stents, and a bare stent connected to the distal end of the stent-graft, wherein, barbs for looseness-proof fixation are respectively provided on the zigzag stent at the proximal end of the stent-graft and on the bare stent. This piece of prior art is mainly applied at the location of the aortic arch, wherein the stent-graft is anchored by the barbs at its proximal end and at its distal end inserting into the blood vessel wall of the aorta, which effectively prevents the migration of the proximal end of the stent-graft body towards the distal end thereof and the migration of the distal end thereof towards the proximal end thereof caused by the transversal force acted on the bended stent-graft by blood flow. However, along with the relaxing-contracting movement of the aorta, the stent-graft and the barbs provided thereon might move relative to the blood vessel wall, and the barbs might detach from the blood vessel wall or become not completely inserted into the blood vessel wall, also, the diameter of aorta of the same individual is not uniform, thus when the selected diameter of the outer periphery of the barbs of the stent-graft does not fit the inner diameter of the aorta at the anchoring location, it is incapable for one set of barbs arranged at the proximal end of the stent-graft to completely anchor the stent-graft, especially, when the stent-graft is applied at the abdominal aorta, because the abdominal aorta is communicated with renal arteries, after the detachment of the stent-graft, the graft thereof might easily block the blood flow between the abdominal aorta and the renal arteries, and thereby might endanger the life of the patient.

### SUMMARY OF THE INVENTION

One objective of the present invention is to solve the technical problem that the anchoring of the looseness-proof fixation structure of the stent-graft in prior art is insecure.

In order to solve the above-mentioned technical problem, the present invention provides an aortic stent-graft, comprising a covered stent body and a waved bare stent connected to a proximal end of the covered stent body, with a first looseness-proof fixation structure provided at each wave crest of the bare stent away from the covered stent body, wherein, a second looseness-proof fixation structure is provided at each wave trough of the bare stent near the covered stent body, the first looseness-proof fixation structure and the second looseness-proof fixation structure extend towards a distal end of the covered stent body.

Preferably, the first looseness-proof fixation structure and the second looseness-proof fixation structure are outspreading barbs, a divergence angle formed between each of the barbs and a central axis of the bare stent is larger than 30°.

Alternatively, two barbs are provided at each wave crest of the bare stent, and an intersection angle is formed between the two barbs.

Alternatively, one barb is provided at each wave crest of the bare stent.

Alternatively, two barbs are provided at each wave trough of the bare stent, and the two barbs are arranged on outer sides of struts on both sides of each wave trough.

Alternatively, one barb is provided at each wave trough of the bare stent, and the barb is arranged on an outer side of a strut on the left side of each wave trough.

Alternatively, one barb is provided at each wave trough of the bare stent, and the barb is arranged on an outer side of a strut on the right side of each wave trough.

Alternatively, one barb is provided at each wave trough of the bare stent, and the barb is arranged on an outer surface of a handle portion for connecting each wave trough to a connection hole.

Preferably, the covered stent body comprises a main stent body, a longer branch stent body and a shorter branch stent body connected together, with the length of the longer branch stent body longer than the length of the shorter branch stent body.

Preferably, the aortic stent-graft is an abdominal aortic stent-graft.

Preferably, the bare stent is provided with an image developing component arranged at a position close to the proximal end of the covered stent body, for monitoring the location of the covered stent body.

Preferably, the image developing component on the bare stent is arranged on at least one of the wave troughs of the bare stent, and is connected to the bare stent by adhesion, inlaying, muff-coupling, soldering or coating.

Preferably, through holes are provided at each wave crest of the bare stent, and a tightening member passes through the through holes to control the bare stent to stretch out and draw back in the radial direction.

Preferably, the tightening member is selected from metal wire, silk thread, cotton thread, plastic wire and flax thread, and the through holes are adapted for the tightening member to pass through and then tighten up, so as to fold the bare stent.

Preferably, connection holes are formed at each wave trough of the bare stent, and a connecting suture passes through each of the connection holes and is connected to the graft membrane at the proximal end of the covered stent body.

Preferably, the covered stent body is further provided with an image developing component thereon for monitoring the location of the covered stent body.

Preferably, an image developing component is further stitched at a position on the graft membrane where the longer branch stent body and the shorter branch stent body are cross connected, for identifying the location of the intersection point of the two branch stent bodies.

Preferably, an image developing component is further stitched respectively at a position on the graft membrane where the longer branch stent body is located and at a position on the graft membrane where the shorter branch stent body is located, for marking the respective location where each branch stent body joins the blood vessel.

Compared to prior art, the present invention has the following advantages:

(1) In the present invention, a second looseness-proof fixation structure is provided at each wave trough of the bare stent near the covered stent body, so that after the aortic stent-graft is fastened by the looseness-proof fixation structures, the second looseness-proof fixation structures at the wave troughs are in coordination with the first looseness-proof fixation structures at the wave crests, and because the second looseness-proof fixation structures at the wave troughs are not on the same plane with the first looseness-proof fixation structures at the wave crests, therefore, along with the relaxing-contracting movement of the aorta, the two sets of looseness-proof fixation structures are not likely to move relative to the blood vessel wall simultaneously to cause detaching or loosening of the looseness-proof fixation structures, thus the aortic stent-graft is able to be fastened onto the blood vessel wall more firmly, and the risk of blocking the renal arteries is further reduced;

(2) In the present invention, the bare stent is provided with a plurality of through holes and a tightening member for controlling the bare stent to stretch out and draw back in the radial direction, the through holes are arranged at each wave crest of the bare stent, thus by using the tightening member to fold the through holes, the bare stent can be controlled to draw back in the radial direction, and then the aortic stent-graft can be compressed into a delivering device with a smaller catheter diameter to be delivered into a human body, this makes it more convenient to fold the stent-graft and put the stent-graft into the delivering device during the operation.

(3) The image developing components are arranged directly on the bare stent, compared to the design of suturing the image developing components on the graft membrane, the volume of the stent-graft after contraction is reduced, so that the required volume of the delivering device is reduced, which facilitates the movement of the delivering device within the aorta and the release of the stent-graft; further, the image developing components are arranged on at least one of the wave troughs of the bare stent, thus are more close to the covered stent body, so as to more precisely indicate the location of the covered stent body.

(4) In the present invention, more image developing components are further connected on the graft membrane respectively near the proximal end of the main stent body, at the intersection point of the two branch stent bodies, and at the position where the two branch stent bodies are respectively located, thus after the aortic stent-graft enters a human body, the respective locations of the main stent body, the intersection point of the two branch stent bodies, as well as the respective two branch stent bodies can be determined by observing the aforementioned image developing components, so as to facilitate the handling of the operation.

### BRIEF DESCRIPTION OF THE DRAWINGS

To make the contents of the present invention more easily understood clearly, further descriptions of the present invention are presented below, in conjunction with specific embodiments and appended drawings, wherein:

FIG. 1 is a schematic diagram of an abdominal aortic stent-graft in Embodiment 1 of the present invention;

FIG. 2 is a schematic diagram of a bare stent of the abdominal aortic stent-graft in Embodiment 1 of the present invention;

FIG. 3 is an unfolded plane schematic diagram of an unstretched bare stent in Embodiment 1 of the present invention;

FIG. 4 is an unfolded plane schematic diagram of an unstretched bare stent in Embodiment 2 of the present invention;

FIG. 5 is an unfolded plane schematic diagram of an unstretched bare stent in Embodiment 3 of the present invention;

FIG. 6 is an unfolded plane schematic diagram of an unstretched bare stent in Embodiment 4 of the present invention;

FIG. 7 is an unfolded plane schematic diagram of an unstretched bare stent in Embodiment 5 of the present invention;

FIG. 8 is an unfolded plane schematic diagram of an unstretched bare stent in Embodiment 6 of the present invention;

FIG. 9 is an unfolded plane schematic diagram of an unstretched bare stent in Embodiment 7 of the present invention;

FIG. 10 is an unfolded plane schematic diagram of an unstretched bare stent in Embodiment 8 of the present invention.

Wherein, the marking numerals are explained as follows:

1-bare stent, 2-main stent body, 3-graft membrane, 4-longer branch stent body, 5-shorter branch stent body, 6-through hole, 7-barb, 8-connection hole, 9-image developing component, 10-strut.

### DETAILED DESCRIPTION OF EMBODIMENTS

### Embodiment 1

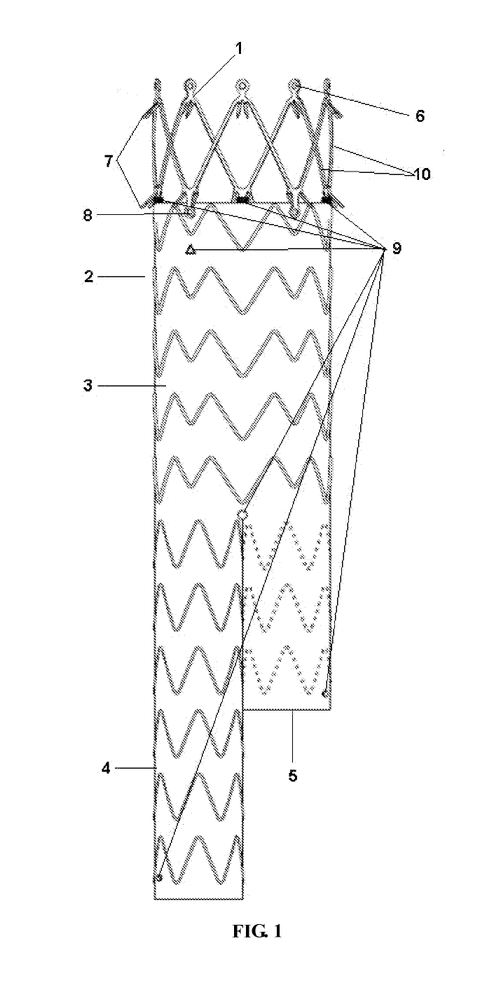

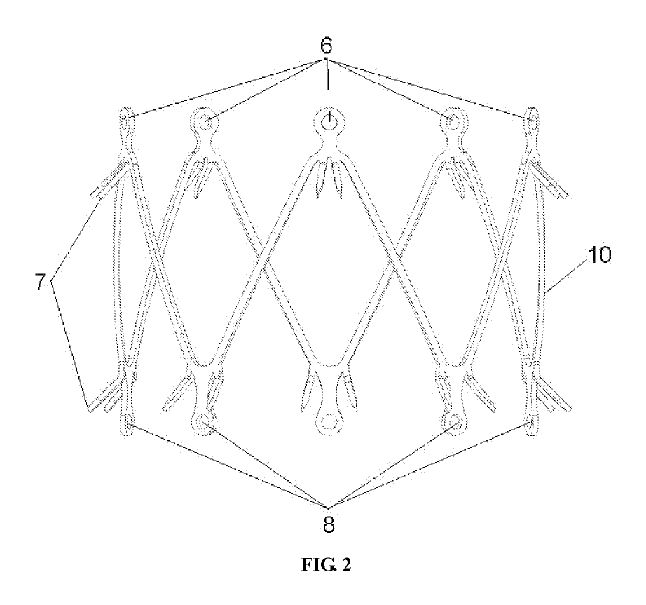

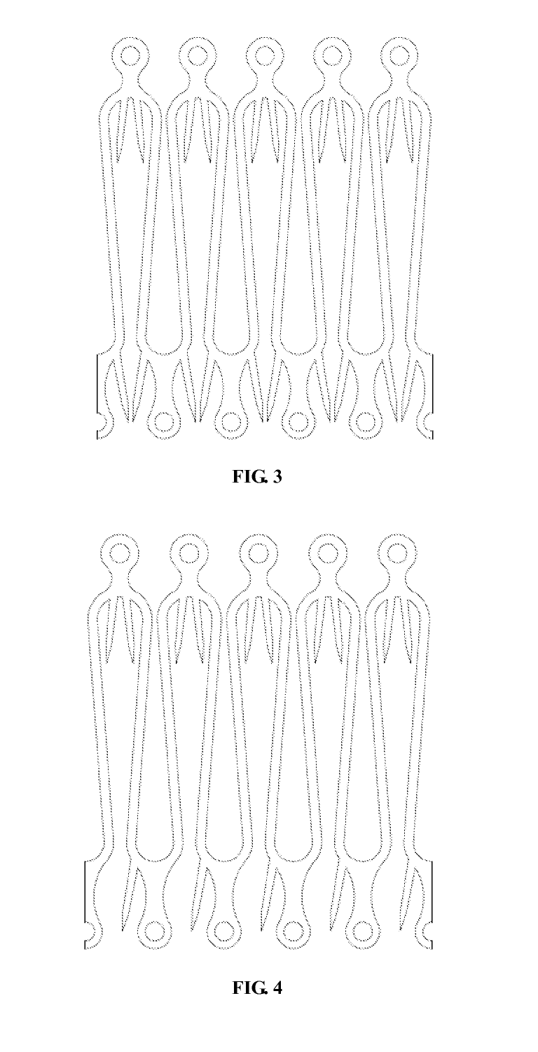

As shown in FIGS. 1-3, an abdominal aortic stent-graft of the first embodiment comprises a main stent body 2 consisting of five waved cylindrical single-circles, a longer branch stent body 4 consisting of six waved cylindrical single-circles, and a shorter branch stent body 5 consisting of three waved cylindrical single-circles; the main stent body 2, the longer branch stent body 4 and the shorter branch stent body 5 are connected together and stitched on a graft membrane 3 with matched shape to form a stent-graft, wherein, the main stent body 2 and the longer branch stent body 4 are respectively sutured on the outer side of the graft membrane 3, while the shorter branch stent body 5 is sutured on the inner side of the graft membrane 3.

The bare stent 1 consists of one waved cylindrical single-circle, with 5 waves, each wave crest of its cylindrical single-circle away from the covered stent body is formed with a through hole 6 for a thread to pass through, so as to tighten or release the volume of the stent-graft; each wave trough of its cylindrical single-circle is formed with a connection hole 8, for a suture to pass through so as to connect the bare stent 1 with the proximal end of the covered stent body; each wave crest of the bare stent 1 away from the covered stent body and each wave trough of the bare stent 1 near the covered stent body are respectively provided with two barbs 7 extending towards the distal end of the covered stent body with a divergence angle of 45°, wherein, the two barbs 7 at each wave crest away from the covered stent body have an intersection angle of 30°, and the two barbs 7 at each wave trough near the covered stent body are arranged on outer sides of struts 10 on both sides of the wave trough, extending towards the distal end of the covered stent body; as further improved embodiments, a bar-shaped image developing component 9 is provided on each of the handle portions of the three consecutive ring-shaped connection holes 8 of the bare stent 1, preferably in a hooped manner or an adhesive manner; a “A” shaped image developing component 9 is further sutured on the graft membrane 3 below the first wave trough (from left to right) of the first cylindrical single-circle from the proximal end of the main stent body 2; an image developing component 9 is further stitched on the graft membrane 3 at the intersection point of the longer branch stent body 4 and the shorter branch stent body 5; the graft membrane 3 at the distal end of the longer branch stent body 4 and the graft membrane 3 at the distal end of the shorter branch stent body 5 further respectively have an image developing component 9 stitched thereon; the material for making all the cylindrical single-circles is Nitinol, and the material for making the graft membrane 3 is polyethylene terephthalate (PET).

### Embodiment 2

The abdominal aortic stent-graft of the second embodiment employs a bare stent 1 as shown in FIG. 4, the bare stent 1 is sutured at each wave trough with the graft membrane 3 at the proximal end of the covered stent body; each wave crest of the bare stent 1 away from the covered stent body is provided with two barbs 7 extending towards the distal end of the covered stent body with a divergence angle of 45°, wherein the two barbs 7 have an intersection angle of 30°, and each wave trough of the bare stent 1 near the covered stent body is provided with one barb 7 arranged on an outer side of a strut on the left side of the wave trough with a divergence angle of 45° and extending towards the distal end of the covered stent body, the structure and the material of other portions are the same as that of Embodiment 1.

### Embodiment 3

The abdominal aortic stent-graft of the third embodiment employs a bare stent 1 as shown in FIG. 5, the bare stent 1 is sutured at each wave trough with the graft membrane 3 at the proximal end of the covered stent body; each wave crest of the bare stent 1 away from the covered stent body is provided with two barbs 7 extending towards the distal end of the covered stent body with a divergence angle of 45°, wherein the two barbs 7 have an intersection angle of 30°, and each wave trough of the bare stent 1 near the covered stent body is provided with one barb 7 arranged on an outer side of a strut on the right side of the wave trough with a divergence angle of 45° and extending towards the distal end of the covered stent body, the structure and the material of other portions are the same as that of Embodiment 1.

### Embodiment 4

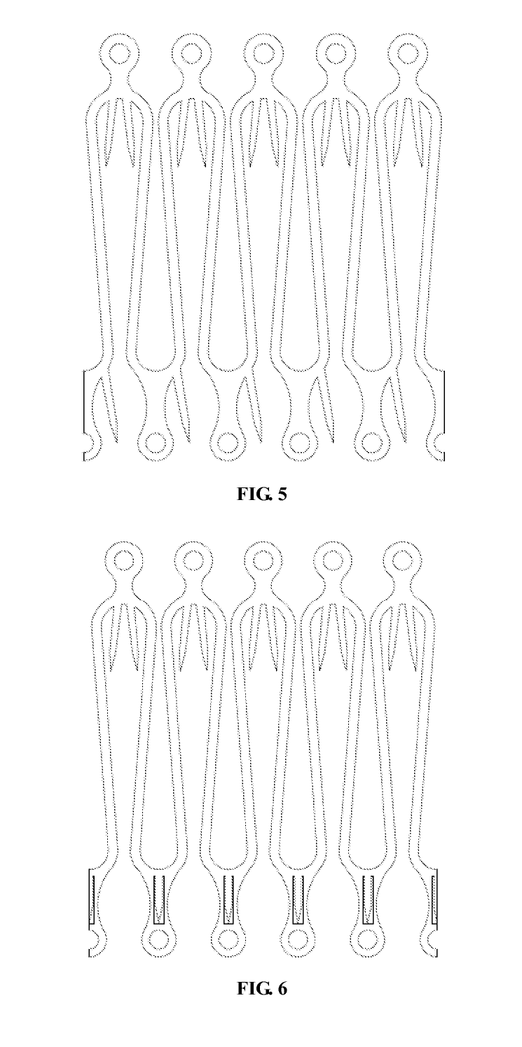

The abdominal aortic stent-graft of the fourth embodiment employs a bare stent 1 as shown in FIG. 6, the bare stent 1 is sutured at each wave trough with the graft membrane 3 at the proximal end of the covered stent body; each wave crest of the bare stent 1 away from the covered stent body is provided with two barbs 7 extending towards the distal end of the covered stent body with a divergence angle of 45°, wherein the two barbs 7 have an intersection angle of 30°, and each wave trough of the bare stent 1 near the covered stent body is provided with one barb 7 arranged on an outer surface of a handle portion for connecting the wave trough to a connection hole 8 with a divergence angle of 45° and extending towards the distal end of the covered stent body, the structure and the material of other portions are the same as that of Embodiment 1.

### Embodiment 5

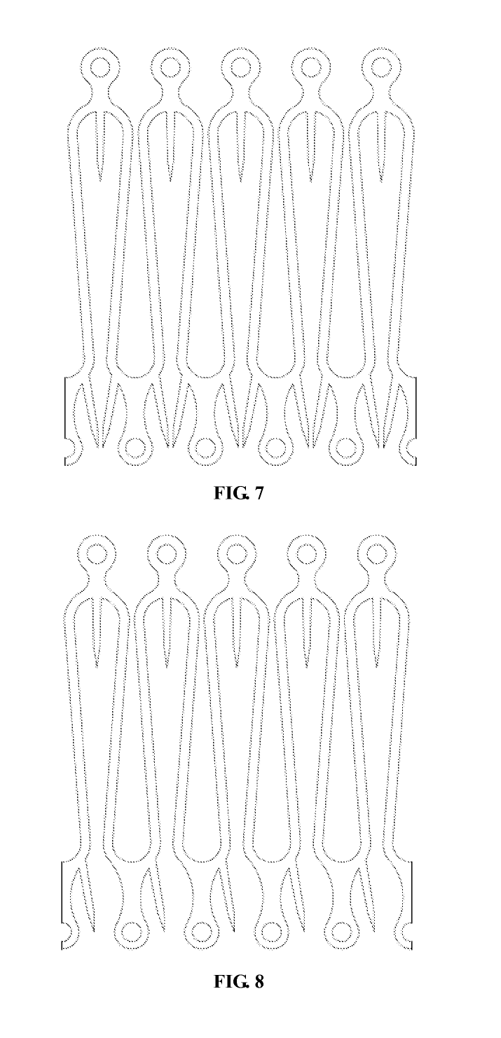

The abdominal aortic stent-graft of the fifth embodiment employs a bare stent 1 as shown in FIG. 7, the bare stent 1 is sutured at each wave trough with the graft membrane 3 at the proximal end of the covered stent body; each wave crest of the bare stent 1 away from the covered stent body is provided with one barb 7 extending towards the distal end of the covered stent body with a divergence angle of 45°, and one barb 7 with a divergence angle of 45° and extending towards the distal end of the covered stent body is provided respectively on each of the two outer sides of struts on both sides of each wave trough of the bare stent 1 near the covered stent body, the structure and the material of other portions are the same as that of Embodiment 1.

### Embodiment 6

The abdominal aortic stent-graft of the sixth embodiment employs a bare stent 1 as shown in FIG. 8, the bare stent 1 is sutured at each wave trough with the graft membrane 3 at the proximal end of the covered stent body; each wave crest of the bare stent 1 away from the covered stent body is provided with one barb 7 extending towards the distal end of the covered stent body with a divergence angle of 45°, and each wave trough of the bare stent 1 near the covered stent body is provided with one barb 7 arranged on an outer side of a strut on the right side of the wave trough with a divergence angle of 45° and extending towards the distal end of the covered stent body, the structure and the material of other portions are the same as that of Embodiment 1.

### Embodiment 7

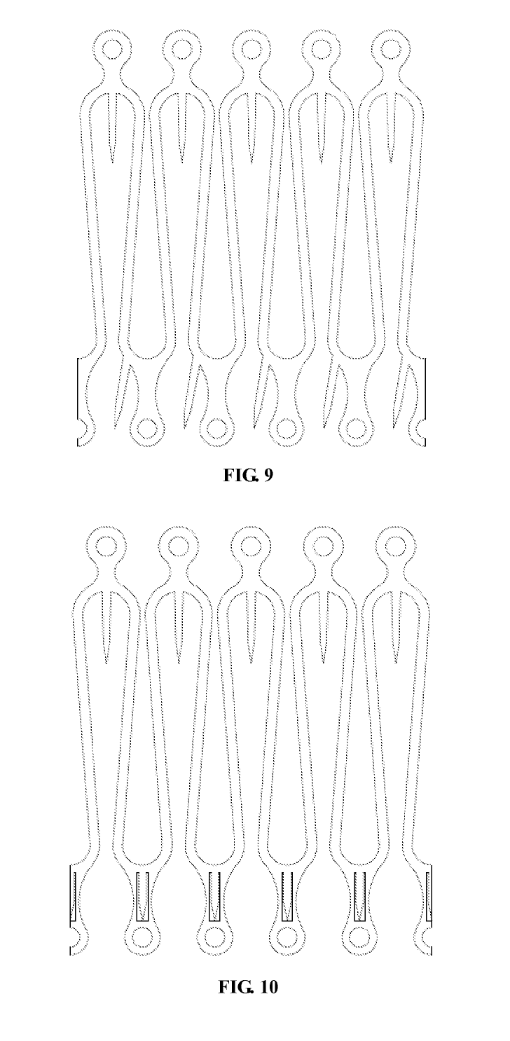

The abdominal aortic stent-graft of the seventh embodiment employs a bare stent 1 as shown in FIG. 9, the bare stent 1 is sutured at each wave trough with the graft membrane 3 at the proximal end of the covered stent body; each wave crest of the bare stent 1 away from the covered stent body is provided with one barb 7 extending towards the distal end of the covered stent body with a divergence angle of 45°, and each wave trough of the bare stent 1 near the covered stent body is provided with one barb 7 arranged on an outer side of a strut on the left side of the wave trough with a divergence angle of 45° and extending towards the distal end of the covered stent body, the structure and the material of other portions are the same as that of Embodiment 1.

### Embodiment 8

The abdominal aortic stent-graft of the eighth embodiment employs a bare stent 1 as shown in FIG. 10, the bare stent 1 is sutured at each wave trough with the graft membrane 3 at the proximal end of the covered stent body; each wave crest of the bare stent 1 away from the covered stent body is provided with one barb 7 extending towards the distal end of the covered stent body with a divergence angle of 45°, and each wave trough of the bare stent 1 near the covered stent body is provided with one barb 7 arranged on an outer surface of a handle portion for connecting the wave trough to a connection hole 8 with a divergence angle of 45° and extending towards the distal end of the covered stent body, the structure and the material of other portions are the same as that of Embodiment 1.

Apparently, the aforementioned embodiments are merely intended for clearly describing the examples, rather than limiting the implementation ways of the invention. For those skilled in the art, various changes and modifications in other different forms can be made on the basis of the aforementioned description. It is unnecessary and impossible to exhaustively list all the implementation ways herein. However, any obvious changes or modifications derived from the aforementioned description are intended to be embraced within the protection scope of the present invention.

1-21. (canceled)

22. An aortic stent-graft, comprising:
a covered stent body; and
a waved bare stent connected to a proximal end of the covered stent body, with a first looseness-proof fixation structure provided at each wave crest of the bare stent away from the covered stent body, wherein,
a second looseness-proof fixation structure is provided at each wave trough of the bare stent near the covered stent body,
the first looseness-proof fixation structure and the second looseness-proof fixation structure extending towards a distal end of the covered stent body are outspreading barbs,
two barbs are provided at each wave crest of the bare stent, and an intersection angle is formed between the two barbs,
the aortic stent-graft is an abdominal aortic stent-graft.

23. The aortic stent-graft of claim 22, wherein, one barb is provided at each wave trough of the bare stent, and the barb is arranged on an outer surface of a handle portion for connecting each wave trough to a connection hole.

24. The aortic stent-graft of claim 22, wherein, the barbs are integrally formed on the bare stent.

25. The aortic stent-graft of claim 22, wherein, a divergence angle formed between each of the barbs and a central axis of the bare stent is larger than 30°.

26. The aortic stent-graft of claim 25, wherein, the divergence angle formed between each of the barbs and the central axis of the bare stent is 45°.

27. The aortic stent-graft of claim 22, wherein, the intersection angle formed between the two barbs at each wave trough of the bare stent is 30°.

28. The aortic stent-graft of claim 22, wherein, the bare stent is provided with an image developing component arranged at a position close to the proximal end of the covered stent body, for monitoring the location of the covered stent body, and the image developing component on the bare stent is arranged on at least one of the wave troughs of the bare stent, above the top edge of the graft membrane and close to the top edge of the graft membrane.

29. The aortic stent-graft of claim 28, wherein, the image developing component is connected to the bare stent by adhesion, inlaying, muff-coupling, soldering or coating.

30. The aortic stent-graft of claims 22, wherein, the covered stent body comprises a main stent body, a longer branch stent body and a shorter branch stent body connected together, with the length of the longer branch stent body longer than the length of the shorter branch stent body.

31. The aortic stent-graft of claims 23, wherein, the covered stent body comprises a main stent body, a longer branch stent body and a shorter branch stent body connected together, with the length of the longer branch stent body longer than the length of the shorter branch stent body.

32. The aortic stent-graft of claims 24, wherein, the covered stent body comprises a main stent body, a longer branch stent body and a shorter branch stent body connected together, with the length of the longer branch stent body longer than the length of the shorter branch stent body.

33. The aortic stent-graft of claims 25, wherein, the covered stent body comprises a main stent body, a longer branch stent body and a shorter branch stent body connected together, with the length of the longer branch stent body longer than the length of the shorter branch stent body.

34. The aortic stent-graft of claims 26, wherein, the covered stent body comprises a main stent body, a longer branch stent body and a shorter branch stent body connected together, with the length of the longer branch stent body longer than the length of the shorter branch stent body.

35. The aortic stent-graft of claims 27, wherein, the covered stent body comprises a main stent body, a longer branch stent body and a shorter branch stent body connected together, with the length of the longer branch stent body longer than the length of the shorter branch stent body.

36. The aortic stent-graft of claims 28, wherein, the covered stent body comprises a main stent body, a longer branch stent body and a shorter branch stent body connected together, with the length of the longer branch stent body longer than the length of the shorter branch stent body.

37. The aortic stent-graft of claims 29, wherein, the covered stent body comprises a main stent body, a longer branch stent body and a shorter branch stent body connected together, with the length of the longer branch stent body longer than the length of the shorter branch stent body.
